(19) United States
(12) Patent Application Publication
(10) Pub. No.: US 2010/0168552 A1
(43) Pub. Date: Jul. 1, 2010

(54) DIPOLE ESTIMATION METHOD

(76) Inventors: Ikuo Homma, Tokyo (JP); Yoshiwo Okamoto, Kawasaki-shi (JP)

Correspondence Address: WENDEROTH, LIND & PONACK, L.L.P., 1030 15th Street, N.W.,, Suite 400 East, Washington, DC, 20005-1503, US

(21) Appl. No.: 12/645,140

(22) Filed: Dec. 22, 2009

(30) Foreign Application Priority Data
Dec. 25, 2008 (JP) 2008-329478

(51) Int. Cl. A61B 5/05

(52) U.S. Cl. 600/409

(57) ABSTRACT

The present invention provides a dipole estimation method which adopts a living body model which is divided into an active area where the conductivity is homogeneous and a passive conductive area having an arbitrary conductivity distribution other than the active area, maintaining the calculation speed at a level as high as that with the conventional art, while allowing the accuracy of potential calculation to be improved.

### TECHNICAL FIELD

The present invention relates to a dipole estimation method, and particularly relates to a dipole estimation method for estimating the location and vector components of a true equivalent dipole in the inside of a living body.

### BACKGROUND ART

The brain in a living body generates an electromotive force from its activity, generating a potential on the scalp. The recording of a change of this potential over time is an electroencephalogram, providing information for clarifying the condition of the brain. Recently, by associating the information of a number of electroencephalograms accumulated over many years with various diseases, the diagnosis of the brain disease has been made possible to a certain degree.

On the other hand, separately from such an empirical approach, there is an attempt to directly estimate the site of activity in the brain from the potential distribution on the head epidermis on the basis of the physical law.

The dipole tracking method is one of the techniques which handle such an inverse problem about brain potential, and it approximates the electromotive force in the brain with several current dipoles, and determines the locations and vector components of the respective dipoles such that the mean squared error between the potential distribution which are generated by them on the head epidermis and the actually measured potential distribution is at a minimum, however, the mean squared error is a complicated nonlinear function for the location of the dipole, which forces use of the repeating method for minimization.

With the dipole tracking method, it is required to slightly change the location of the dipole while repetitively calculating the potential distribution, thus the calculation of the potential distribution must be performed at a sufficiently high speed.

As conventional examples of an efficient potential calculation method for dipole estimation, the inventions disclosed in Japanese Patent Laid-Open Publication No. 6-22916, Japanese Patent Laid-Open Publication No. 7-213501, and Japanese Patent Laid-Open Publication No. 8-257004 have been proposed, and they will be described hereinbelow.

With the invention disclosed in Japanese Patent Laid-Open Publication No. 6-22916, the skull is approximated as a piecewise homogeneous conductor; the boundary element method is used to derive a discrete equation concerning the potential at the boundary face and the normal derivative thereof for each division; all the divisions are bound under a boundary condition to derive a whole equation; and by solving this, the potential which is generated by the dipole on the scalp is calculated.

Finally, a matrix is calculated which binds the potential which is generated by the dipole on the surface of the brain in an unlimitedly homogeneous medium the entire space of which is filled with a medium having the same conductivity as that of the brain and the normal derivative thereof, with the potential on the scalp. In other words, when N nod es are disposed on the surface of the brain for discretization, if the N-dimensional vector produced by arranging the potentials which are generated by the dipoles at these nodes in the unlimitedly homogeneous medium is u₁^{∞}, and the N-dimensional vector produced by arranging the normal derivatives of the potentials is q₁^{∞}, the M-dimensional vector u_{E} produced by arranging the potentials at the M electrodes disposed on the surface of the piecewise uniform skull is given as the following formula in [Math 1], as shown in the formula “ [symbol] ” (re) in Japanese Patent Laid-Open Publication No. 6-22916.

$${u}_{E} = {B}_{E} ( \begin{matrix}{u}_{1}^{∞} \\ {q}_{1}^{∞}\end{matrix} ) \qquad [ Math 1 ]$$

Here, the matrix B_{E} of M rows and 2N columns is established by the shape and the conductivity distribution of the skull model and the electrode location on the scalp independently of the location and vector components of the dipole, thus it can be calculated once for every subject. The amount of calculation for calculating the matrix B_{E} is relatively large, however, once the matrix B_{E} becomes known, calculation of the potential distribution can be performed at an extremely high speed. This is because the potentials u₁^{∞} which are generated by the dipoles in an unlimitedly homogeneous medium and the normal derivatives thereof q₁^{∞} can be extremely easily calculated, and simply by causing the matrix B_{E} to act on the 2N-dimensional vector produced by arranging those, the potential distribution u_{E} on the scalp can be calculated.

With the invention disclosed in Japanese Patent Laid-Open Publication No. 7-213501, the skull is approximated as a piecewise homogeneous conductor of three-layer structure consisting of a brain, a skull, and a scalp. As a skull model, this invention is only a special application of the invention disclosed in the Japanese Patent Laid-Open Publication No. 6-22916, however, such speciality is utilized to realize a still higher efficiency in potential calculation.

In other words, with the invention disclosed in Japanese Patent Laid-Open Publication No. 6-22916, a whole equation involving all the divisions is derived, and an equation which makes the potentials at the discrete points on all the boundaries and the normal derivatives thereof to be unknowns is handled, however, with the invention disclosed in Japanese Patent Laid-Open Publication No. 7-213501, the speciality due to the skull model having a layered structure is utilized to eliminate the unknowns for every division for reducing the scale of the equation, whereby the amount of calculation is reduced.

Finally, a result corresponding to the formula in [Math 1] is obtained, however, a fact that q₁^{∞} is in linear relationship with u₁^{∞} is indicated, and by utilizing this for eliminating q₁^{∞}, the following formula in [Math 2] is obtained.

u_{E}=Tu₁^{∞} [Math 2]

Eventually, in order to calculate the potential distribution u_{E} on the scalp, it is only required to cause the matrix of M rows and N columns to act on the N-dimensional vector produced by arranging the potentials which are generated by the dipoles at the N nodes on the surface of the brain in an unlimitedly homogeneous medium, which reduces the amount of calculation to less than one half of that required when the formula in [Math 1] is used.

The aforementioned formula in [Math 2] corresponds to the formula (23) in Japanese Patent Laid-Open Publication No. 7-213501, where u₁^{∞}, u_{E}, and T are denoted by u′_{b}, uₛ, M, respectively.

A technique with which the skull model of three-layer structure is replaced with that of four-layer structure is proposed in Japanese Patent Laid-Open Publication No. 8-257004. In other words, by providing an brain cerebrospinal fluid area between the brain and the skull, the skull is approximated as a piecewise homogeneous conductor of four-layer structure.

As a skull model, this is also only a special application of the invention disclosed in the Japanese Patent Laid-Open Publication No. 6-22916, however, the speciality of layered structure is utilized to eliminate the unknowns for every division for reducing the scale of the equation, whereby a higher efficiency is realized.

The formula (21) as the final result that is given in Japanese Patent Laid-Open Publication No. 8-257004 is the same as formula (23), including the notation, in the invention disclosed in Japanese Patent Laid-Open Publication No. 7-213501 that is applicable to three-layer skull models, however, needless to say, the potential transfer matrix M corresponds to the four-layer skull model.

As aforedescribed, with conventional equivalent dipole methods, the human skull, for example, is approximated by using a three-layer skull model consisting of a scalp, a skull, and a brain (Japanese Patent Laid-Open Publication No. 7-213501), or a four-layer skull model consisting of the same plus a brain cerebrospinal fluid layer (Japanese Patent Laid-Open Publication No. 8-257004).

The invention disclosed in Japanese Patent Laid-Open Publication No. 6-22916 does not always premise a layered structure, but for this reason, it is restricted also in terms of making high speed potential calculation. In any of the aforementioned inventions, it is assumed that the conductivity distribution in the skull is piecewise homogeneous.

However, especially in the vicinity of the skull base and the eye pit, the skull inside has an extremely complicated structure, thus there is a limitation in approximating this as a piecewise homogeneous conductor. In other words, when the dipole is located in the vicinity of the skull base or the eye pit, the accuracy of potential calculation is lowered.

[Patent document 1] Japanese Patent Laid-Open Publication No. 6-22916
[Patent document 2] Japanese Patent Laid-Open Publication No. 7-213501
[Patent document 3] Japanese Patent Laid-Open Publication No. 8-257004

### DISCLOSURE OF THE INVENTION

### Problems to be Solved by the Invention

The present invention provides a dipole estimation method which adopts a living body model which is divided into an active area where the conductivity is homogeneous (for example, brain tissue) and a passive conductive area having an arbitrary conductivity distribution other than the active area, maintaining the calculation speed at a level as high as that with the conventional art, while allowing the accuracy of potential calculation to be improved.

### Means for Solving the Problems

The dipole estimation method pertaining to the present invention provides a dipole estimation method for estimating the location and vector components of a dipole 2 in the inside of a skull model Mo by using the measured value of a potential on the surface of the skull model Mo for performing a predetermined calculation, the dipole estimation method comprising the steps of dividing the inside of the skull model Mo into an active area Ω_{B} having a homogeneous conductivity, containing a dipole, and a passive conductive area Ω_{O} having an arbitrary conductivity distribution other than the active area Ω_{B}; previously calculating a potential transfer matrix from a potential distribution which would be generated on the surface of the active area Ω_{B} if a plurality of dipoles were given in an unlimitedly homogeneous medium, to potentials at a plurality of electrodes loaded on the surface of the skull model Mo; measuring the potential distribution on the surface of the skull model Mo by means of the plurality of electrodes 1 loaded on the surface of the skull model Mo; calculating the potentials at a plurality of electrodes 1 loaded on the surface of the skull model Mo, by multiplying a potential distribution which would be generated on the surface of the active area if a plurality of dipoles 2 were given in an unlimitedly homogeneous medium, by the aforementioned potential transfer matrix; and estimating the true location and vector components of the dipole 2 in the active area Ω_{B} by repetitively modifying the locations and vector components of the plurality of dipoles 2 previously set in the active area Ω_{B} such that the squared error between the calculated potential distribution on the surface of the skull model Mo and the measured potential distribution on the surface of the skull model Mo is at a minimum.

The dipole estimation method pertaining to the present invention provides, as a most important feature, a dipole estimation method for estimating the location and vector components of a dipole in the inside of a living body by using the measured value of a potential on the surface of the living body for performing a predetermined calculation, the dipole estimation method comprising the steps of dividing the inside of the living body into an active area having a homogeneous conductivity, containing a dipole, and a passive conductive area having an arbitrary conductivity distribution other than the active area; previously calculating a potential transfer matrix from a potential distribution which would be generated on the surface of the active area if a plurality of dipoles were given in an unlimitedly homogeneous medium, to potentials at a plurality of electrodes loaded on the surface of the living body; measuring the potential distribution on the surface of the living body by means of the plurality of electrodes loaded on the surface of the living body; calculating the potentials which are generated by the dipoles temporarily set in the active area at the electrodes on the surface of the living body, by multiplying a potential distribution which would be generated on the surface of the active area if these dipoles were given in an unlimitedly homogeneous medium, by the aforementioned potential transfer matrix; and estimating the true location and vector components of the dipole in the active area by repetitively modifying the locations and vector components of the plurality of dipoles set in the active area such that the squared error between the calculated potential distribution on the surface of the living body and the measured potential distribution is at a minimum.

### ADVANTAGES OF THE INVENTION

According to the invention as stated in Claim 1, the inside of a living body is divided into an active area having a homogeneous conductivity, and a passive conductive area having an arbitrary conductivity distribution other than the active area; on the basis of the shapes and conductivity distributions thereof and the locations of electrodes on the surface of the living body, a potential transfer matrix (a matrix which associates a potential distribution which, if a dipole were given in an unlimitedly homogeneous medium, would be generated on the surface of the active area, with a potential distribution on the surface of an actual living body) is previously calculated; then the potential distribution on the surface of the living body (scalp) which is generated by the dipole in the active area is measured; on the basis of information about the temporary locations and the temporary vector components of a plurality of dipoles temporarily set in the active area, the potential distribution on the surface of the living body is calculated using the potential transfer matrix; and the true location and vector components of the dipole in the active area are estimated by correcting the locations and vector components of the plurality of dipoles set in the active area such that the squared error between the calculated potential distribution on the surface of the living body and the measured potential distribution is at a minimum. Thereby, the calculation speed for dipole estimation is maintained at a level as high as that with the conventional art, potential calculation concerning the active area in the inside of a living body that is closer to the reality and higher in accuracy can be realized.

According to the invention as stated in Claim 2, even when a part of the passive conductive area exists in the inside of the active area, potential calculation concerning the active area in the inside of a living body that is closer to the reality and higher in accuracy can be realized as with the invention as stated in Claim 1.

According to the invention as stated in Claim 3, a scheme is adopted which comprises steps similar to those in the invention as stated in Claim 2, and previously calculates a potential transfer matrix from potentials which would be generated at a plurality of nodes on the surface of the active area if a plurality of dipoles were given in an unlimitedly homogeneous medium, to potentials at a plurality of electrodes loaded on the surface of the living body, whereby potential calculation concerning the active area in the inside of a living body that is closer to the reality and higher in accuracy can be realized as with the invention as stated in Claim 1.

### BEST MODE FOR CARRYING OUT THE INVENTION

It is an object of the present invention to provide a dipole estimation method which adopts a living body model which is divided into an active area where the conductivity is homogeneous (for example, brain tissue) and a passive conductive area having an arbitrary conductivity distribution other than the active area, maintaining the calculation speed at a level as high as that with the conventional art, while allowing the accuracy of potential calculation to be improved.

The present invention has achieved the aforementioned object with a dipole estimation method for estimating the location and vector components of a dipole in the inside of a living body by using the measured value of a potential on the surface of the living body for performing a predetermined calculation, the dipole estimation method comprising the steps of dividing the inside of the living body into an active area having a homogeneous conductivity, containing a plurality of dipoles, and a passive conductive area having an arbitrary conductivity distribution other than the active area, including a case where a part of the passive conductive area exists in the inside of the active area; previously calculating a potential transfer matrix from potentials which would be generated at a plurality of nodes on the surface of the active area if a plurality of dipoles were given in an unlimitedly homogeneous medium, to potentials at a plurality of electrodes loaded on the surface of the living body; measuring the potential distribution on the surface of the living body by means of the plurality of electrodes loaded on the surface of the living body; and estimating the true location and vector components of the dipole in the active area by correcting the locations and vector components of the plurality of dipoles set in the active area such that the squared error between the calculated potential distribution on the surface of the living body and the measured potential distribution is at a minimum, and repeating this correction.

### Embodiment

Hereinbelow, a dipole estimation method pertaining to an embodiment of the present invention will be described in details with reference to the drawings.

The dipole estimation method pertaining to the present embodiment takes the steps required for dipole estimation that can be divided into the three broad general categories:

(1) creating a living body model (for example, a skull model Mo), and on the basis thereof, calculating a potential transfer matrix (a matrix which associates a potential distribution which, if a dipole were given in an unlimitedly homogeneous medium, would be generated on the surface of the active area, with a potential distribution on the surface of an actual living body);
(2) measuring the living body potential (the potential distribution on the surface of the living body at a number of sample times, for example, the change in brain potential over time); and
(3) using the potential transfer matrix to perform the dipole estimation at the respective sample times.

More specifically, the dipole estimation method pertaining to the present embodiment is constituted by the following steps.

As shown in FIG. 5, the inside of a living body, such as a skull, is divided into an active area having a homogeneous conductivity and contains a plurality of dipoles, and a passive conductive area having an arbitrary conductivity distribution other than the active area, including a case where a part of the passive conductive area exists in the inside of the active area (step S1); a matrix for potential transfer from the potentials at a plurality of node set on the surface of the active area to the potentials at a plurality of electrodes loaded on the surface of the living body is calculated (step S2); the potential distribution on the surface of the living body is measured by means of the plurality of electrodes loaded on the surface of the living body (step S3); the locations and vector components of the plurality of dipoles previously set in the active area are corrected such that the squared error between the calculated potential distribution on the surface of the living body and the actually measured potential distribution is at a minimum (step S4); and by repeating this correction, the true location and vector components of the dipole in the active area are estimated (step S5).

Hereinbelow, the specific process contents for realizing the processes in the aforementioned step S1 to step S5 of the dipole estimation method pertaining to the present embodiment will be described in details.

FIG. 1 is a conceptual drawing of the skull model Mo in actual shape that is adopted in the dipole estimation method pertaining to the present embodiment. In FIG. 1, the active area Ω_{B} containing a dipole 2 (shown with an arrow), and having a homogeneous conductivity is shown with a white color, while the passive conductive areas Ω_{O} and Ω_{I} having an arbitrary conductivity distribution other than the active area are shown with a gray color.

As shown in FIG. 1, a part of the passive conductive area Ω_{O} (the passive conductive area Ω_{I}) may be surrounded by the active area Ω_{B}. In addition, in FIG. 1, the number of passive conductive areas Ω_{I} which are surrounded by the active area Ω_{B} is 3, however, actually, there may be any number thereof.

Further, as the active area Ω_{B}, it may be the entire brain parenchyma, or may be limited to a part of the brain parenchyma. If the latter is adopted, the non-uniformity in conductivity in the part of the brain can be reflected. In FIG. 1, reference numeral 1 denotes an electrode disposed on the surface of the living body, and reference numeral 2 denotes a dipole.

In addition, as shown in FIG. 2, the passive conductive area which is surrounded by the active area Ω_{B} of the skull model Mo is denoted by Ω_{I}, and the passive conductive area which surrounds the active area Ω_{B} is denoted by Ω_{O}.

The boundary face between the active area Ω_{B} and the passive conductive area Ω_{I} is denoted by Γ_{BI}; the boundary face between the active area Ω_{B} and the passive conductive area Ω_{O} is denoted by Γ_{BO}; and the surface of the active area Ω_{B} and the passive conductive area Ω_{O}, in other words, the surface of the living body is denoted by Γ_{O}.

Further, the number of nodes set on the boundary faces Γ_{BI} and Γ_{BO}, and the surface of the living body Γ_{O} for discretization are denoted by N_{BI}, N_{BO}, and N_{O}, respectively (nodes are also provided in the inside of the passive conductive areas Ω_{I} and Ω_{O}, however, in the following description, no reference characters which denote the number of internal nodes will be used).

Since no electromotive force (dipole 2) is contained in the inside of the passive conductive area Ω_{O}, if the potential is specified at the boundary face Γ_{BO}, and the normal component of the current density is specified at the boundary face Γ_{O}, the potential distribution in the passive conductive area Ω_{O} is established, hence, the normal component of the current density at the boundary face Γ_{BO}, and the potential at the boundary face Γ_{O} are also established.

Therefore, if N_{BO}-dimensional vectors produced by arranging the normal components of the potential and current density at the N_{BO} nodes on the boundary face Γ_{BO} are u_{BO} and j_{BO}, and N_{O}-dimensional vectors produced by arranging the normal components of the potential and current density at the N_{O} nodes on the boundary face Γ_{O} are u_{O} and j_{O}, the j_{BO} and u_{O} are established as linear functions of the u_{BO} and j_{O}, and the following formula in [Math 3] and the following formula in [Math 4], which is obtained by rewriting the formula in [Math 3], are given.

$$\begin{matrix}\{ \begin{matrix}{j}_{BO} = {T}_{BO , BO}^{O} {u}_{BO} + {T}_{BO , O}^{O} {j}_{O} \\ {u}_{O} {T}_{O , BO}^{O} {u}_{BO} + {T}_{O , O}^{O} {j}_{O}\end{matrix} & [ Math 3 ] \\ ( \begin{matrix}{j}_{BO} \\ {u}_{O}\end{matrix} ) = {T}^{O} ( \begin{matrix}{u}_{BO} \\ {j}_{O}\end{matrix} ) , {T}^{O} = ( \begin{matrix}{T}_{BO , BO}^{O} & {T}_{BO , O}^{O} \\ {T}_{O , BO}^{O} & {T}_{O , O}^{O}\end{matrix} ) & [ Math 4 ]\end{matrix}$$

In this formula, the subscripts BO and O in the vectors u_{BO}, j_{BO}, u_{O}, and j_{O} indicate that the value is that on the boundary face Γ_{BO} and the surface of the living body Γ_{O}, respectively, and the superscript “O” in the matrices T_{BO,BO}^{O}, T_{BO,O}^{O}, T_{O,BO}^{O}, T_{O,O}^{O}, and T^{O} means that the amount is that concerning the passive conductive area Ω_{O}.

In addition, the sets of subscripts in the matrices T_{BO,BO}^{O}, T_{BO,O}^{O}, T_{O,BO}^{O}, and T_{O,O}^{O} indicate that between which boundaries the respective matrices connect. For example, T_{O,BO}^{O} is a transfer factor from the potential distribution u_{BO} on the boundary face Γ_{BO} to the potential distribution u_{O} on the surface of the living body Γ_{O}.

In any event, for determining the (N_{BO}+N_{O})th-order square matrix T^{O}, various numerical calculation techniques can be utilized depending upon the case.

For example, if the conductivity distribution in the passive conductive area Ω_{O} is completely arbitrary, use of the finite element method is realistic, while when the passive conductive area Ω_{O} is approximated as the piecewise uniform system, the boundary element method is effective.

Since no electromotive force is contained inside the passive conductive area Ω_{I}, if the potential is specified at the boundary face Γ_{BI}, the potential distribution in the passive conductive area Ω_{I} is established, hence, the normal component of the current density at the boundary face Γ_{BI} is also established.

Therefore, if N_{BI}-dimensional vectors produced by arranging the normal components of the potential and current density at the N_{BI} nodes on the boundary face Γ_{BI} are u_{BI} and j_{BI}, respectively, the j_{BI} is established as a linear function of the u_{BI}, and the following formula in [Math 5] is given.

j_{BI}=T^{I}u_{BI} [Math 5]

In this formula, the subscript “BI” in the vector u_{BI}, j_{BI} means that the value is that on the boundary face Γ_{BI}, and the superscript “I” in the T^{I} indicates that the amount is that concerning the passive conductive area Ω_{I}.

In addition, as in calculating the T^{O}, the N_{I}th-order square matrix can be calculated by using the boundary element method or the finite element method depending upon whether the passive conductive area Ω_{I} is piecewise uniform.

On the other hand, the active area Ω_{B} contains the dipole 2, however, the conductivity σ_{B} is constant in the active area Ω_{B}. Consequently, if the potential distribution which is generated by the dipole 2 in the active area Ω_{B} is φ, and the potential distribution which is generated by the same dipole 2 in the unlimitedly homogeneous medium having a conductivity σ_{B} is φ^{∞}, the φ−φ^{∞} meets the Laplace equation as given by the following formula in [Math 6] in the active area Ω_{B}.

∇²(φ−φ^{∞})=0 [Math 6]

Therefore, by using the boundary element method concerning the Laplace equation, the following formula in [Math 7] is obtained.

$$( \begin{matrix}{H}_{BI , BI} & {H}_{BI , BO} \\ {H}_{BO , BI} & {H}_{BO , BO}\end{matrix} ) ( \begin{matrix}{u}_{BI} \\ {u}_{BO}\end{matrix} ) = ( \begin{matrix}{G}_{BI , BI} & {G}_{BI , BO} \\ {G}_{BO , BI} & {G}_{BO , BO}\end{matrix} ) ( \begin{matrix}{q}_{BI} \\ {q}_{BO}\end{matrix} ) + ( \begin{matrix}{u}_{BI}^{∞} \\ {u}_{BO}^{∞}\end{matrix} ) \qquad [ Math 7 ]$$

In this formula, the u_{BI} and u_{BI} are N_{BI}-dimensional and N_{BO}-dimensional vectors produced by arranging the values of the potentials φ at N_{BI} nodes on the boundary face Γ_{BI}, and N_{BO} nodes on the boundary face Γ_{BO}, respectively, and the q_{BI} and q_{BO} are N_{BI}-dimensional and N_{BO}-dimensional vectors produced by arranging the normal derivatives of the potentials at the nodes on the boundary face Γ_{BI}, and the nodes on the boundary face Γ_{BO}, respectively.

In addition, the u_{BI}^{∞} and u_{BO}^{∞} are N_{BI}-dimensional and N_{BO}-dimensional vectors produced by arranging the values of the φ^{∞} at the nodes on the boundary face Γ_{BI} and the nodes on the boundary face Γ_{BO}, respectively.

However, since the normal components q_{BI} and q_{BO} of the potential, and the normal components j_{BI} and j_{BO} of the current density have a relationship as given in the following formula in [Math 8], the aforementioned formula in [Math 7] can be expressed by the following formula in [Math 9], and by further rewriting the formula in [Math 9], the following formula in [Math 10] is given. In the formula in [Math 8] and the formula in [Math 9], the σ_{B} is the conductivity of the active area Ω_{B}.

$$\begin{matrix} {j}_{BI} = - {σ}_{B} {q}_{BI} , {j}_{BO} = - {σ}_{B} {q}_{BO} & [ Math 8 ] \\ ( \begin{matrix}{H}_{BI , BI} & {H}_{BI , BO} \\ {H}_{BO , BI} & {H}_{BO , BO}\end{matrix} ) ( \begin{matrix}{u}_{BI} \\ {u}_{BO}\end{matrix} ) = \frac{- 1}{{σ}_{B}} ( \begin{matrix}{G}_{BI , BI} & {G}_{BI , BO} \\ {G}_{BO , BI} & {G}_{BO , BO}\end{matrix} ) ( \begin{matrix}{j}_{B I} \\ {j}_{BO}\end{matrix} ) + ( \begin{matrix}{u}_{BI}^{∞} \\ {u}_{BO}^{∞}\end{matrix} ) & [ Math 9 ] \\ \{ \begin{matrix}{H}_{BI , BI} {u}_{BI} + {H}_{BI , BO} {u}_{BO} = - {σ}_{B}^{- 1} ( {G}_{BI , BI} {j}_{BI} + {G}_{BI , BO} {j}_{BO} ) + {u}_{BI}^{∞} \\ {H}_{BO , BI} {u}_{BI} + {H}_{BO , BO} {u}_{BO} = - {σ}_{B}^{- 1} ( {G}_{BO , BI} {j}_{BI} + {G}_{BO , BO} {j}_{BO} ) + {u}_{BO}^{∞}\end{matrix} & [ Math 10 ]\end{matrix}$$

If the j_{BI} and j_{BO} appearing on the right side of the formula in [Math 10] are expressed with the u_{BI}, u_{BO}, and j_{O}, using the formula in [Math 5] and the formula in [Math 3], respectively, the following formula in [Math 10a] and the following formula in [Math 10b] as a result of rearrangement of the formula in [Math 10a] are obtained, and finally the following formula in [Math 11] is derived.

$$\begin{matrix}\{ \begin{matrix}{H}_{BI , BI} {u}_{BI} + {H}_{BI , BO} {u}_{BO} = - {σ}_{B}^{- 1} \{ {G}_{BI , BI} {T}^{I} {u}_{BI} + {G}_{BI , BO} ( {T}_{BO , BO}^{O} {u}_{BO} + {T}_{BO , O}^{O} {j}_{O} ) \} + {u}_{BI}^{∞} \\ {H}_{BO , BI} {u}_{BI} + {H}_{BO , BO} {u}_{BO} = - {σ}_{B}^{- 1} \{ {G}_{BO , BI} {T}^{I} {u}_{BI} + {G}_{BO , BO} ( {T}_{BO , BO}^{O} {u}_{BO} + {T}_{BO , O}^{O} {j}_{O} ) \} + {u}_{BO}^{∞}\end{matrix} & [ Math 10 a ] \\ \{ \begin{matrix}( {H}_{BI , BI} + {σ}_{B}^{- 1} {G}_{BI , BI} {T}^{I} ) {u}_{BI} + ( {H}_{BI , BO} + {σ}_{B}^{- 1} {G}_{BI , BO} {T}_{BO , BO}^{O} ) {u}_{BO} = - {σ}_{B}^{- 1} {G}_{BI , BO} {T}_{BO , O}^{O} {j}_{O} + {u}_{BI}^{∞} \\ ( {H}_{BO , BI} + {σ}_{B}^{- 1} {G}_{BO , BI} {T}^{I} ) {u}_{BI} + ( {H}_{BO , BO} + {σ}_{B}^{- 1} {G}_{BO , BO} {T}_{BO , BO}^{O} ) {u}_{BO} = - {σ}_{B}^{- 1} {G}_{BO , BO} {T}_{BO , O}^{O} {j}_{O} + {u}_{BO}^{∞}\end{matrix} & [ Math 10 b ] \\ P ( \begin{matrix}{u}_{BI} \\ {u}_{BO}\end{matrix} ) = - {σ}_{B}^{- 1} ( \begin{matrix}{G}_{BI , BO} \\ {G}_{BO , BO}\end{matrix} ) {T}_{BO , O}^{O} {j}_{O} + ( \begin{matrix}{u}_{BI}^{∞} \\ {u}_{BO}^{∞}\end{matrix} ) & [ Math 11 ]\end{matrix}$$

However, the (N_{BI}+N_{BO})th-order square matrix P is defined as the following formula in [Math 12].

$$P = ( \begin{matrix}{H}_{BI , BI} + {σ}_{B}^{- 1} {G}_{BI , BI} {T}^{I} & {H}_{BI , BO} + {σ}_{B}^{- 1} {G}_{BI , BO} {T}_{BO , BO}^{O} \\ {H}_{BO , BI} + {σ}_{B}^{- 1} {G}_{BO , BI} {T}^{I} & {H}_{BO , BO} + {σ}_{B}^{- 1} {G}_{BO , BO} {T}_{BO , BO}^{O}\end{matrix} ) \qquad [ Math 12 ]$$

Here, when the (N_{BI}+N_{BO})-dimensional vector with which all the components are 1 is “e”, the reference point of potential can be arbitrarily selected, whereby the following formula in [Math 13] is given, and thus the square matrix P is not reversible.

Pe=0, e=(1 1 . . . 1)^{t} [Math 13]

However, if the j_{O} meets the physical conditions, in other words, if the whole current flowing in from the surface of the living body Γ_{O} (which can be expressed as the inner product between an appropriate N_{O}-dimensional vector w and the e) is zero, the aforementioned formula in [Math 11] has a solution. This solution has an ambiguity providing solutions the number of which is a constant multiple of the e, and, one of the solutions is given as the following formula in [Math 14], using the Moore-Penrose general matrix inverse P⁺ for the square matrix P.

$$( \begin{matrix}{u}_{BI} \\ {u}_{BO}\end{matrix} ) = - {σ}_{B}^{- 1} {P}^{+} ( \begin{matrix}{G}_{BI , BO} \\ {G}_{BO , BO}\end{matrix} ) {T}_{BO , O}^{O} {j}_{O} + {P}^{+} ( \begin{matrix}{u}_{BI}^{∞} \\ {u}_{BO}^{∞}\end{matrix} ) \qquad [ Math 14 ]$$

In performing a dipole estimation, the reference of potential is established such that the average of the potentials of all the electrode is zero, the ambiguity of the solution has no effect on the dipole estimation, thus, if the solution of the formula in [Math 14] is adopted, the generality will not be lost.

Here, if the general matrix inverse P⁺ is divided into the first N_{BI} row and the remaining N_{BO} row to be expressed as the following formula in [Math 15], the u_{BO} on the left side of the formula in [Math 14] is given as the following formula in [Math 16], and by substituting this into the formula in [Math 3], the following formula in [Math 17] is obtained.

$$\begin{matrix} {P}^{+} = ( \begin{matrix}{P}_{BI}^{+} \\ {P}_{BO}^{+}\end{matrix} ) & [ Math 15 ] \\ {u}_{BO} = - {σ}_{B}^{- 1} {P}_{BO}^{+} ( \begin{matrix}{G}_{BI , BO} \\ {G}_{BO , BO}\end{matrix} ) {T}_{BO , O}^{O} {j}_{O} + {P}_{BO}^{+} ( \begin{matrix}{u}_{BI}^{∞} \\ {u}_{BO}^{∞}\end{matrix} ) & [ Math 16 ] \\ {u}_{O} = {T}_{O , BO}^{O} {P}_{BO}^{+} ( \begin{matrix}{u}_{BI}^{∞} \\ {u}_{BO}^{∞}\end{matrix} ) + \{ {T}_{O , O}^{O} - {σ}_{B}^{- 1} {T}_{O , BO}^{O} {P}_{BO}^{+} ( \begin{matrix}{G}_{BI , BO} \\ {G}_{BO , BO}\end{matrix} ) {T}_{BO , O}^{O} \} {j}_{O} & [ Math 17 ]\end{matrix}$$

This means that the potential distribution u_{O} on the surface of the living body Γ_{O} is determined by the conduction current j_{O} from the surface of the living body Γ_{O} and the potential distributions u_{BI}^{∞} and u_{BO}^{∞} in the surface of the active area Ω_{B} in the unlimitedly homogeneous medium.

By the way, the surface of the living body Γ_{O} is normally an insulating boundary, and thus the conduction current j_{O} is j_{O}=0. Hence, the aforementioned formula in [Math 17] can normally be written as the following formula in [Math 18].

$${u}_{O} = {T}_{O , BO}^{O} {P}_{BO}^{+} {u}_{B}^{∞} , {u}_{B}^{∞} = ( \begin{matrix}{u}_{BI}^{∞} \\ {u}_{BO}^{∞}\end{matrix} ) \qquad [ Math 18 ]$$

In this manner, the potentials u_{O} at the N_{O} nodes set on the surface of the living body Γ_{O} are calculated, however, what is finally required is not the potentials u_{O} at the nodes, but the potentials at the respective locations of the respective electrodes 1 disposed on the surface of the living body Γ_{O}.

When the potentials at the respective locations of the respective electrodes 1 are to be calculated by linear interpolation of the node potentials, the M-dimensional vector u_{E} produced by arranging the M electrode potentials can be written as the following formula in [Math 19], using a matrix W of M rows and N_{O} columns expressing the interpolation calculation, and by substituting the formula in [Math 18] into the right side of the formula in [Math 19], the following formula in [Math 20] is obtained.

u_{E}=Wu_{O} [Math 19]

u_{E}=Tu_{B}^{∞}, T=WT_{O,BO}^{O}P_{BO}⁺ [Math 20]

This is formally the same as the formula (2) which has been obtained in the aforementioned Japanese Patent Laid-Open Publication No. 7-213501 and Japanese Patent Laid-Open Publication No. 8-257004, however, the formula (2) provides a result from a three-layer or four-layer piecewise homogeneous skull model, the formula in [Math 20] in the present embodiment provides a result from a system having an arbitrary conductivity distribution.

In this manner, the present invention maintains the calculation speed at a level as high as that with the conventional art, while allowing highly accurate potential calculation to be made for the active area Ω_{B} in the precision skull model Mo which is still closer to the reality.

Next, how to make the matrix T^{O} calculation more efficient and accurate will be described.

The active area Ω_{B} is the entire brain parenchyma or a part thereof, and the passive conductive area Ω_{O} surrounding it has a layered structure. In other words, around the brain, there exist the brain cerebrospinal fluid and the brain and spinal cord membranes, and surrounding the outside thereof, the skull, and further the scalp and face exist. This is the reason why a skull model having a three-layer or four-layer is conventionally used.

Especially, the skull has an extremely low conductivity compared to the other tissue, thus when the potential is to be numerically calculated, it is desirable that the skull be handled separately from the other tissue. Conventionally, a piecewise uniform model which assumes the conductivity in the respective layer to be constant has been adopted, however, for example, the conductivity of the skull varies depending upon the site in accordance with the difference in bone density, and the like, and also in the scalp and face, the conductivity varies in accordance with the tissue, such as the muscle and fat.

Then, in the present embodiment, the skull model Mo with which the passive conductive area Ω_{O} is divided into layered structures, but the conductivity of the respective layers is not always homogeneous is adopted, and hereinbelow, a method for efficiently calculating the (N_{BO}+N_{O})th square matrix T^{O} which is defined by the aforementioned formula in [Math 4] will be described.

FIG. 3 illustrates the case where the passive conductive area Ω_{O} is constituted by piecewise homogeneous areas Ω₂, Ω₃, . . . , and Ω_{N} in N−1 layers. The conductivity of the areas Ω₂, Ω₃, . . . , Ω_{N} is assumed to be σ₂, σ₃, . . . , σ_{N}, respectively.

For uniform notation, the boundary face Γ_{BO} inside the area Ω₂ shows n in FIG. 3 is denoted by Γ₁, while the boundary face Γ_{O} outside the area Ω_{N} is denoted by Γ_{N}. In addition, the boundary face between the area Ω₂ and the area Ω₃ is denoted by Γ₂, the same manner as this being taken in the following, and lastly, the boundary face between the area Ω_{N-1} and the area Ω_{N} is denoted by Γ_{N-1}.

As shown in FIG. 3, the normal direction of the boundary face Γₙ (n=2˜N−1) is selected to be the direction from the area Ω_{N} toward the area Ω_{N-1}, and the normal directions of the boundary face Γ₁ and the boundary face Γ_{N} are selected to be the direction from the outside of the area Ω₂ tow ard the inside, and the direction from the inside of the area Ω_{N} tow ard the outside, respectively.

Further, for discretization, the number of nodes set on the boundary face Γₙ (n=1˜N) is assumed to be Nₙ (n=1˜N), respectively, and the Nₙ-dimensional vectors produced by arranging the normal components of the potential and current density at the Nₙ nodes on the boundary face Γₙ are denoted by uₙ and jₙ, respectively.

Since no electromotive force exists in the area Ωₙ (n=1˜N), if the potential distribution uₙ₋₁ is specified on the boundary face Γₙ₋₁, and the normal component jₙ of the current density is given on the boundary face Γ_{N}, the potential distribution in the area Ω_{N} is established, and especially, the normal component jₙ₋₁ of the current density on the boundary face Γ_{N-1}, and the potential distribution uₙ on the boundary face Γ_{N} are determined.

Therefore, the jₙ₋₁ and uₙ are established as linear functions of the uₙ₋₁ and jₙ, and the following formula in [Math 21] and formula in [Math 22] are given.

$$\begin{matrix}\{ \begin{matrix}{j}_{n - 1} = {T}_{n - 1 , n - 1}^{n} {u}_{n - 1} + {T}_{n - 1 , n}^{n} {j}_{n} \\ {u}_{n} = {T}_{n , n - 1}^{n} {u}_{n - 1} + {T}_{n , n}^{n} {j}_{n}\end{matrix} & [ Math 21 ] \\ ( \begin{matrix}{j}_{n - 1} \\ {u}_{n}\end{matrix} ) = {T}^{n} ( \begin{matrix}{u}_{n - 1} \\ {j}_{n}\end{matrix} ) , \\ {T}^{n} = ( \begin{matrix}{T}_{n - 1 , n - 1}^{n} & {T}_{n - 1 , n}^{n} \\ {T}_{n , n - 1}^{n} & {T}_{n , n}^{n}\end{matrix} ) & [ Math 22 ]\end{matrix}$$

Here, the superscript “n” in the matrices Tⁿ and T_{n-1,n}ⁿ means that the amount is that concerning the area Ωₙ.

In the formula in [Math 22], if the n is replaced with the n+1, the relational expression for the area Ωₙ₊₁ is obtained, and if this is combined with the formula in [Math 22], the unknown uₙ and jₙ concerning the boundary face Γₙ can be eliminated, and the following formula in [Math 23] is obtained.

$$( \begin{matrix}{j}_{n - 1} \\ {u}_{n + 1}\end{matrix} ) = {T}^{n \sim n + 1} ( \begin{matrix}{u}_{n - 1} \\ {j}_{n + 1}\end{matrix} ) , \\ {T}^{n \sim n + 1} = ( \begin{matrix}{T}_{n - 1 , n - 1}^{n \sim n + 1} & {T}_{n - 1 , n + 1}^{n \sim n + 1} \\ {T}_{n + 1 , n - 1}^{n \sim n + 1} & {T}_{n + 1 , n + 1}^{n \sim n + 1}\end{matrix} ) \qquad [ Math 23 ]$$

However, in the formula in [Math 23], a relationship of the following formula in [Math 24] is involved, and in the formula in [Math 24], the Iₙ is an Nₙ th-order unit matrix.

$$\{ \begin{matrix}{T}_{n - 1 , n - 1}^{n \sim n + 1} = {T}_{n - 1 , n - 1}^{n} + {T}_{n - 1 , n}^{n} {Q}_{n} {T}_{n , n}^{n + 1} {T}_{n , n - 1}^{n} \\ {T}_{n - 1 , n + 1}^{n \sim n + 1} = {T}_{n - 1 , n}^{n} {Q}_{n} {T}_{n , n + 1}^{n + 1} \\ {T}_{n + 1 , n - 1}^{n \sim n + 1} = {T}_{n + 1 , n}^{n + 1} {P}_{n} {T}_{n , n - 1}^{n} \\ {T}_{n + 1 , n + 1}^{n \sim n + 1} = {T}_{n + 1 , n}^{n + 1} {P}_{n} {T}_{n , n}^{n} {T}_{n , n + 1}^{n + 1} + {T}_{n + 1 , n + 1}^{n + 1} ,\end{matrix} \\ \{ \begin{matrix}{P}_{n} = {( {I}_{n} - {T}_{n , n}^{n} {T}_{n , n}^{n + 1} )}^{- 1} \\ {Q}_{n} = {( {I}_{n} - {T}_{n , n}^{n + 1} {T}_{n , n}^{n} )}^{- 1}\end{matrix} \qquad [ Math 24 ]$$

As shown in FIG. 4, the formula in [Math 23] gives an input/output relationship in the area Ωₙ∪Ωₙ₊₁ which is produced by merging two areas Ωₙ and Ωₙ₊₁.
To incorporate this fact, the “n˜n+1” is used as the superscript in the T^{n˜n+1} and T_{n+1,n-1}^{n˜n+1}.

In any event, by repeating the same operation, the areas from Ω₂ to Ω_{N} can be merged, all the variables concerning the respective boundary faces from the boundary faces Γ₂ to Γ_{N-1} being eliminated, and the following formula in [Math 25] being given.

$$( \begin{matrix}{j}_{1} \\ {u}_{N}\end{matrix} ) = {T}^{2 \sim N} ( \begin{matrix}{u}_{1} \\ {j}_{N}\end{matrix} ) , \\ {T}^{2 \sim N} = ( \begin{matrix}{T}_{1 , 1}^{2 \sim N} & {T}_{1 , N}^{2 \sim n} \\ {T}_{N , 1}^{2 \sim N} & {T}_{N , N}^{2 \sim N}\end{matrix} ) ( \begin{matrix}{u}_{1} \\ {j}_{N}\end{matrix} ) \qquad [ Math 25 ]$$

However, from the relationship of the following formula in [Math 26], the formula in [Math 27] is given, whereby the formula in [Math 25] means the following formula in [Math 28], and as can be seen from comparison of this with the aforementioned formula in [Math 4], the following formula in [Math 29] is obtained. In this manner, a matrix required for the passive conductive area Ω_{O} can be efficiently calculated using a computer apparatus.

$$\begin{matrix}{Ω}_{2} ⋃ {Ω}_{3} ⋃ … ⋃ {Ω}_{N} = {Ω}_{O} , {Γ}_{1} = {Γ}_{BO} , {Γ}_{N} = {Γ}_{O} & [ Math 26 ] \\ {u}_{1} = {u}_{BO} , {j}_{1} = {j}_{BO} , {u}_{N} = {u}_{O} , {j}_{N} = {j}_{O} & [ Math 27 ] \\ ( \begin{matrix}{j}_{BO} \\ {u}_{O}\end{matrix} ) = {T}^{2 \sim N} ( \begin{matrix}{u}_{BO} \\ {j}_{O}\end{matrix} ) & [ Math 28 ] \\ {T}^{2 \sim N} = {T}^{O} & [ Math 29 ]\end{matrix}$$

When the area Ωₙ (n=2 to N) is all homogeneous, in other words, the passive conductive area Ω₀ is a piecewise uniform system having a layered structure, the transfer matrix for each area can be efficiently calculated by the calculation process with a computer apparatus using the boundary element method.

When a part of the area Ωₙ (n=2˜N) is homogeneous, and the remaining is non-homogeneous, the transfer matrix for each area can be efficiently calculated by the calculation process with a computer apparatus using the boundary element method for the former, and the finite element method for the latter.

Even when the area Ωₙ (n=2˜N) is all non-homogeneous, the aforementioned technique which divides the passive conductive area Ω_{O} into several small areas is efficient as compared to the technique which handles the entire passive conductive area Ω_{O} at once by the finite element method.

### INDUSTRIAL APPLICABILITY

The present invention is widely applicable to diagnosis of brain diseases, pathological researches, clinical researches, and the like.

### BRIEF DESCRIPTION OF THE DRAWINGS

FIG. 1 is a conceptual drawing illustrating a skull model in real shape that is adopted in a dipole estimation method pertaining to an embodiment of the present invention;

FIG. 2 is a conceptual drawing illustrating a skull model in real shape (containing a passive conductive area surrounded by an active area) that is adopted in the dipole estimation method pertaining to the embodiment of the present invention;

FIG. 3 is a conceptual drawing illustrating a skull model in real shape (in which the active area is comprised of a piecewise homogeneous area having N−1 layers) that is adopted in the dipole estimation method pertaining to the embodiment of the present invention;

FIG. 4 is an explanatory drawing illustrating an input/output relationship in the area which is produced by merging two areas in the dipole estimation method pertaining to the embodiment of the present invention; and

FIG. 5 is a flowchart illustrating processing steps in the dipole estimation method pertaining to the embodiment of the present invention.

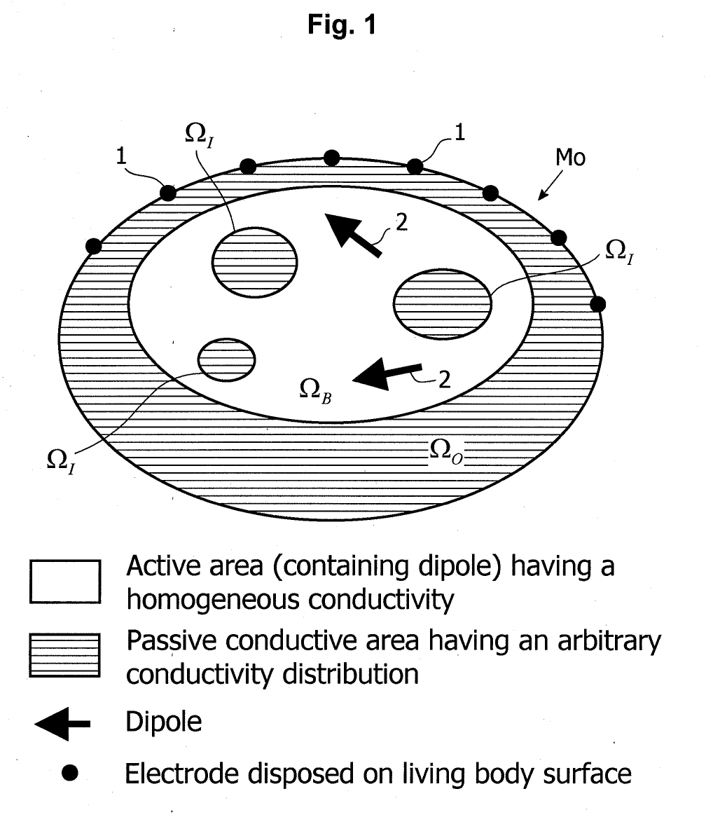

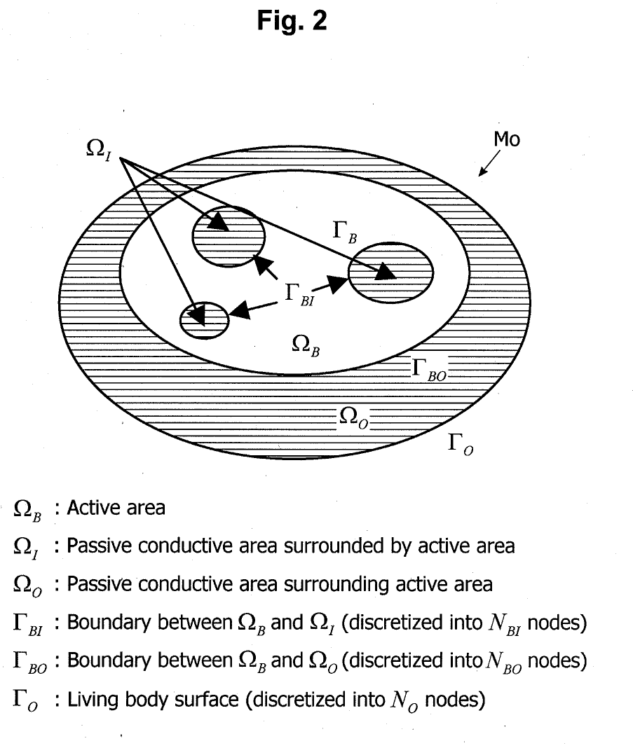

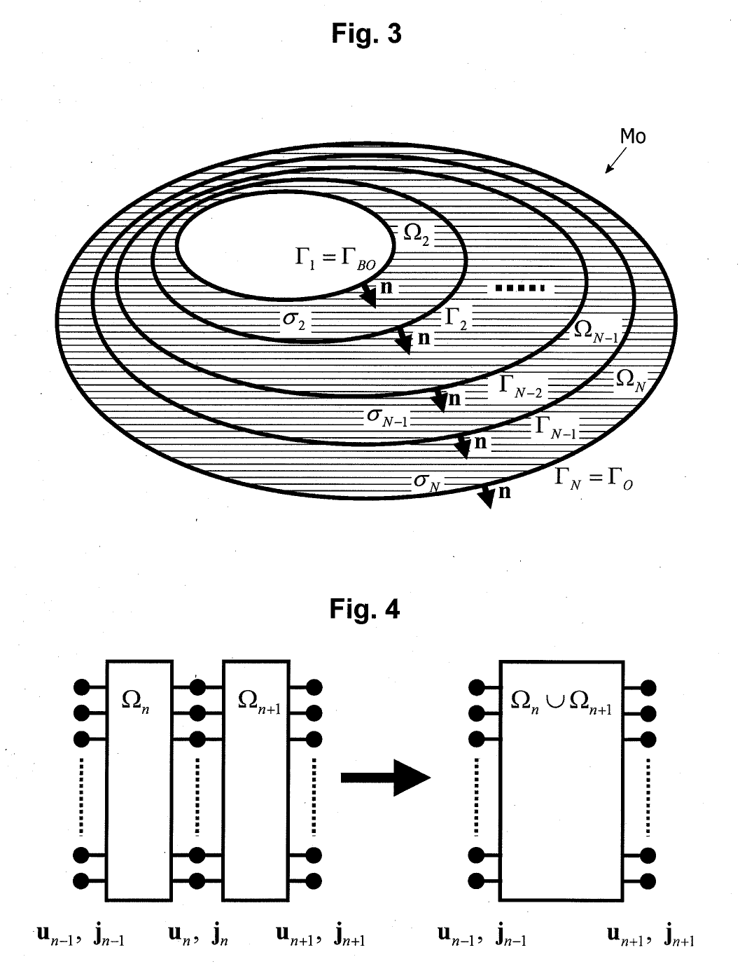

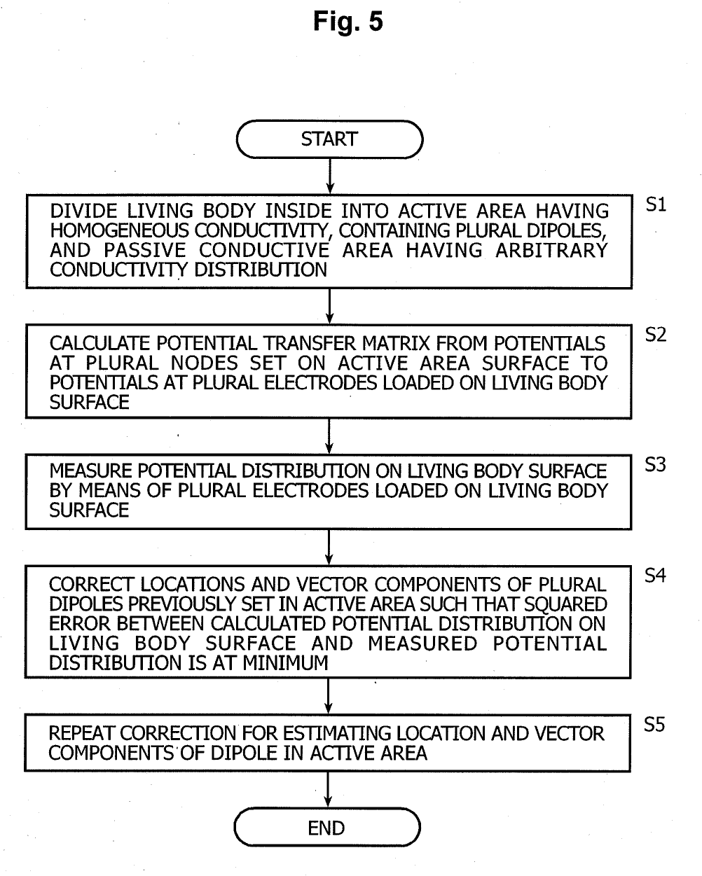

### DESCRIPTION OF SYMBOLS

1: Electrode
2: Dipole
Mo: Skull model
Ω_{B}: Active area
Ω_{O}: Passive conductive area
Ω_{I}: Passive conductive area
Γ_{BI}: Boundary face
Γ_{BO}: Boundary face
Γ_{O}: Surface of living body
Γ₁: Boundary face
Γ_{N}: Boundary face
Ω₂, Ω₃, . . . , Ω_{N}: Area
σ₂, σ₃, . . . , σ_{N}: Conductivity

1. A dipole estimation method for estimating the location and vector components of a dipole in the inside of a living body by using the measured value of a potential on the surface of the living body for performing a predetermined calculation, the dipole estimation method comprising the steps of:
dividing the inside of the living body into an active area having a homogeneous conductivity, containing a dipole, and a passive conductive area having an arbitrary conductivity distribution other than the active area;
previously calculating a potential transfer matrix from a potential distribution which would be generated on the surface of the active area if a plurality of dipoles were given in an unlimitedly homogeneous medium, to potentials at a plurality of electrodes loaded on the surface of said living body;
measuring the potential distribution on the surface of the living body by means of the plurality of electrodes loaded on the surface of said living body;
calculating the potentials which are generated by the dipoles temporarily set in the active area at the electrodes on the surface of the living body, by multiplying a potential distribution which would be generated on the surface of the active area if these dipoles were given in an unlimitedly homogeneous medium, by the aforementioned potential transfer matrix; and
estimating the true location and vector components of the dipole in said active area by repetitively modifying the locations and vector components of the plurality of dipoles set in said active area such that the squared error between the calculated potential distribution on the surface of the living body and the measured potential distribution is at a minimum.

2. A dipole estimation method for estimating the location and vector components of a dipole in the inside of a living body by using the measured value of a potential on the surface of the living body for performing a predetermined calculation, the dipole estimation method comprising the steps of
dividing the inside of the living body into an active area having a homogeneous conductivity, containing a plurality of dipoles, and a passive conductive area having an arbitrary conductivity distribution other than the active area, including a case where a part of the passive conductive area exists in the inside of the active area;
previously calculating a potential transfer matrix from a potential distribution which would be generated on the surface of the active area if a plurality of dipoles were given in an unlimitedly homogeneous medium, to potentials at a plurality of electrodes loaded on the surface of said living body;
measuring the potential distribution on the surface of the living body by means of the plurality of electrodes loaded on the surface of said living body;
calculating the potentials which are generated by the dipoles temporarily set in the active area at the electrodes on the surface of the living body, by multiplying a potential distribution which would be generated on the surface of the active area if these dipoles were given in an unlimitedly homogeneous medium, by the aforementioned potential transfer matrix; and
estimating the true location and vector components of the dipole in said active area by repetitively modifying the locations and vector components of the plurality of dipoles set in said active area such that the squared error between the calculated potential distribution on the surface of the living body and the measured potential distribution is at a minimum.

3. A dipole estimation method for estimating the location and vector components of a dipole in the inside of a living body by using the measured value of a potential on the surface of the living body for performing a predetermined calculation, the dipole estimation method comprising the steps of:
dividing the inside of the living body into an active area having a homogeneous conductivity, containing a plurality of dipoles, and a passive conductive area having an arbitrary conductivity distribution other than the active area, including a case where a part of the passive conductive area exists in the inside of the active area;
previously calculating a potential transfer matrix from potentials which would be generated at a plurality of nodes on the surface of the active area if a plurality of dipoles were given in an unlimitedly homogeneous medium, to potentials at a plurality of electrodes loaded on the surface of said living body;
measuring the potential distribution on the surface of the living body by means of the plurality of electrodes loaded on the surface of said living body;
calculating the potentials which are generated by the dipoles temporarily set in the active area at the electrodes on the surface of the living body, by multiplying a potential distribution which would be generated on the surface of the active area if these dipoles were given in an unlimitedly homogeneous medium, by the aforementioned potential transfer matrix; and
estimating the true location and vector components of the dipole in said active area by correcting the locations and vector components of the plurality of dipoles set in said active area such that the squared error between the calculated potential distribution on the surface of the living body and the measured potential distribution is at a minimum.
